(19) United States
(12) Patent Application Publication
(10) Pub. No.: US 2006/0195098 A1
(43) Pub. Date: Aug. 31, 2006

(54) Implant system and fastening element for an implant system

(76) Inventor: Jorg Schumacher, Tuttlingen (DE)

Correspondence Address: RATNERPRESTIA, P O BOX 980, VALLEY FORGE, PA, 19482-0980, US

(21) Appl. No.: 11/358,900

(22) Filed: Feb. 21, 2006

(30) Foreign Application Priority Data
Feb. 22, 2005 (DE) 10 2005 009 282.9

(51) Int. Cl. A61F 2/30

(52) U.S. Cl. 606/61

(57) ABSTRACT

The present invention relates to a fastening element for an implant system, which comprises at least two fastening elements fixable to bone parts or the like and at least one connecting element fixable to the at least two fastening elements, wherein the fastening element comprises a fastening part with a fastening portion at the distal end as well as a receiving part connected to the fastening part, wherein the receiving part comprises a connecting-element receiver for receiving the connecting element as well as a fixing element for fixing the fastening element in the connecting-element receiver, wherein the connecting-element receiver is designed asymmetrically in relation to an axis of symmetry or longitudinal axis of the fixing element.

The present disclosure relates to the subject matter disclosed in German patent application 10 2005 009 282.9 of Feb. 22, 2005, which is incorporated herein by reference in its entirety and for all purposes.

### BACKGROUND OF THE INVENTION

The present invention relates to a fastening element for an implant system, which comprises at least two fastening elements fixable to bone parts or the like and at least one connecting element fixable to the at least two fastening elements, wherein the fastening element comprises a fastening part with a fastening portion at the distal end as well as a receiving part connected to the fastening part, wherein the receiving part comprises a connecting-element receiver for receiving the connecting element as well as a fixing element for fixing the fastening element in the connecting-element receiver.

The present invention further relates to an implant system having at least two fastening elements fixable to bone parts or the like as well as at least one connecting element fixable to the two fastening elements.

To treat damage of the vertebral column in the region of the cervical or lumbar vertebrae, vertebral column stabilizing systems are used in the form of implant systems of the initially described type, which comprise fastening elements of the initially described type in the form of bone screws. One example of a known bone screw is disclosed in U.S. Pat. No. 5,672,176. The bone screw described there has a spherical-segment-shaped head for a so-called “tulip-shaped” receiving part, which is supported pivotably on the head and may receive in a slot a connecting element in the form of a rod. For fixing the rod, a nut is provided, which may be screwed onto an external thread of the receiving part. The use of such bone screws is problematical in the region of the cervical vertebrae since, because of the design of the articulated connection between the fastening portion and the receiving part of the bone screw, a pivoting angle is limited on all sides. Because of the small dimensions of the cervical vertebrae, the screws do however have to be pivoted in one direction, generally upwards.

A further bone screw that is optimized for use in the region of the cervical vertebrae is known from EP 1 205 153 B1. The bone screw described there has in the bottom edge of the receiving part a countersunk portion to allow the fastening part of the bone screw to be pivoted a little further relative to the receiving part in a turning position of the receiving part relative to the fastening part. The drawback of this development is that, when the implant system is inserted, extreme care has to be taken regarding the way in which the fastening parts of the bone screws are screwed into the vertebral bodies. Deviations in the orientation of the fastening part may at worst lead to the receiving part, because of the shape of the connecting element, being seated in a way that precisely prevents further pivoting in the desired manner.

Therefore, it would be preferable to improve a fastening element and an implant system of the initially described type in such a way that a fixing of the connecting element to the fastening element is improved.

### SUMMARY OF THE INVENTION

In a fastening element of the initially described type, it is advantageous according to the invention that the connecting-element receiver is designed asymmetrically in relation to an axis of symmetry or longitudinal axis of the fixing element.

The development according to the invention makes it possible to fix the connecting element, for example a rod, in the receiving part, and namely in such a way that the connecting element is held on the receiving part asymmetrically in relation to the fixing element. Thus, for example in the case of an articulated connection between receiving part and fastening part, upon pivoting of the receiving part relative to the fastening part the fastening part may be pivoted further in a preferred direction even when a connection of the receiving part and the fastening part is designed symmetrically. Furthermore, by virtue of the asymmetrical design of the connecting-element receiver, even if the fastening part is not positioned exactly according to requirements, in the case of an articulated connection between receiving part and fastening part it is still possible to utilize almost the completely enlarged pivoting angle range. In other words, even a rotation of the receiving part about its longitudinal axis, for example, does not lead to a suddenly large reduction of the pivoting angle range, as in the case of a screw known from EP 1 205 153 B1, but merely to a negligible reduction of the pivoting angle range. Designing the connecting-element receiver asymmetrically in relation to an axis of symmetry or longitudinal axis of the fixing element moreover has the advantage that an introduction of force via the fixing element to the connecting element and the connecting element receiver is effected likewise asymmetrically. Thus, not only are forces transmitted in the direction of the longitudinal axis or axis of symmetry of the fixing element to the connecting element and the connecting-element receiver, but also components transversely of the longitudinal axis or axis of symmetry of the fixing element. This leads in particular to a marked improvement of a clamping of the connecting element between the fixing element and the connecting-element receiver, in contrast to fastening elements with a symmetrical introduction of force.

It is advantageous when the connecting-element receiver is inclined by a connecting-element angle of inclination relative to a reference plane extending transversely of an axis of symmetry or longitudinal axis of the fixing element. As a result of this development, the connecting element fixed in the connecting-element receiver is likewise inclined relative to the reference plane. The angle of inclination may lie for example in a range of 2° to 60°. Because of the inclination, when a receiving part is aligned axially relative to the fastening part this already allows a relative position between the connecting element and the fastening part that, in the case of conventional screws, already requires a pivoting of the receiving part relative to the fastening part. The extra mobility of the fastening part relative to the receiving part makes it possible in particular to preset an even greater pivoting angle range.

It is advantageous when the receiving part, for forming the connecting-element receiver, has a substantially U-shaped cross section with two free limbs, which are provided with a fixing thread, and when the U-shaped cross section is formed by one inclined slot, or by two slots of differing depth, between the free limbs. Slots of differing depth between the free limbs produce an inclined connecting-element receiver, into which a connecting element, for example a connecting rod may be inserted, so that said element is likewise in an inclined position relative to the reference plane. Otherwise, the design of the receiving part in this form is particularly simple. Thus, for example, conventional receiving parts may be exchanged or even developed in the manner according to the invention by enlarging a slot.

It is advantageous when the fastening part and the receiving part are connected immovably to one another. In this way, the stability of the fastening element may be increased.

According to a preferred form of construction of the invention, it may however also be provided that the fastening part in an adjustment position is adjustable relative to the receiving part and in a fixing position is fixable relative to the receiving part. This development allows a relative position between the fastening part and the receiving part to be adjusted and fixed in a desired manner. In this way, orthopaedically related, complicated connection situations may be solved.

It is advantageous when the fastening part and the receiving part are connected in an articulated manner to one another. An articulated connection allows the fastening part and the receiving part to be supplied already in a preassembled state, thereby minimizing the risk of losing one of the two parts during surgery. An adjustment of the two parts relative to one another is moreover facilitated.

So that a desired position between the fastening part and the receiving part may be adjusted, it is advantageous when an articulated connection is provided between a proximal end of the fastening part and a distal end of the receiving part and when the receiving part at the distal end has a receiver for the proximal end of the fastening part. The fastening part and the receiving part are therefore easily connectable in articulated manner to one another. For example, the joint may take the form of a hinged joint or bail joint.

It is particularly advantageous when the articulated connection is designed in the form of a ball joint connection and when the proximal end of the fastening part is designed in the form of a spherical-segment-shaped head and the receiver is designed in the form of a dome-shaped seat. In this way, it is possible to pivot the receiving part relative to the fastening part in the adjustment position, namely about a joint centre in the centre of the spherical-segment-shaped head. At the same time, the ball joint connection also allows a rotation of the receiving part about its longitudinal axis, so that by means of the fastening element according to the invention even a not optimally positioned fastening part may be connected in a desired manner to a connecting element.

According to a preferred form of construction of the invention, it may be provided that the receiving part comprises a fixing thread that interacts with a fixing-element thread of the fixing element.

Advantageously, the fixing thread is an internal thread or an external thread and the fixing element is a screw or a nut. The connecting element may therefore be fastened particularly easily to the receiving part. Both types of thread are simple to manufacture, wherein the internal thread may interact with a screw as a fixing element, the external thread with a nut.

Advantageously, the receiving part has on its proximal end an open first bore and on the opposite end to the first bore a second bore, the diameter of which is larger than the diameter of a distal end of the fastening part and smaller than the diameter of the proximal end of the fastening part. This development allows the fastening part to be inserted by its distal end through the second bore, wherein it is ensured that the fastening part does not pass completely through the second bore because the diameter of the proximal end of the fastening part is larger than the diameter of the second bore.

The receiving part is particularly simple to manufacture when the edge delimiting the free end of the second bore is designed symmetrically, viewed in relation to a longitudinal axis of the first bore. This moreover allows a relative movement between the receiving part and the fastening part in an identical manner in all directions.

The receiving part is preferably of a substantially sleeve-shaped design. It is therefore easy and economical to manufacture.

So that the connecting element is fixable in an optimum manner in the connecting-element receiver, it is advantageous when the fixing element has a tip oriented in distal direction and when the tip in the fixing position has an outer surface that is inclined by an angle of inclination relative to the reference plane. In the most advantageous case, the fastening element may be designed in such a way that the outer surface is in line- or surface contact with the connecting element, with the result that damage of the connecting element and of the fixing element may be reduced. By virtue of the inclined outer surface it is possible to transmit an asymmetrical introduction of force from the fixing element directly to the connecting element or indirectly via a clamping element to the connecting-element receiver. This may advantageously result in additional clamping because of transverse forces parallel to the reference plane.

It is particularly advantageous when a portion or area of the outer surface extends parallel or substantially parallel to the connecting-element receiver. In this way, the connecting element is pressed by the fixing element parallel to the connecting-element receiver into the latter and held in the fixing position.

It is advantageous when the connecting-element angle of inclination corresponds to the angle of inclination. In this way, the fixing element may be in line- or surface contact with the connecting element, which lies parallel to the outer surface in the connecting-element receiver.

The angle of inclination advantageously has a value in the range of 3° to 60°. An enlarged pivoting angle of at most 60° is therefore adjustable compared to conventional screws. Even better stability values are achieved when the angle of inclination has a value in the range of 5° to 30°.

So that a position of the fixing element may easily be varied, it is advantageous when the fixing element has a fixing-element tool receiver.

The fastening part advantageously has a tool receiver disposed on the proximal end. Thus, by means of a tool, for example a screw-driving tool, the fastening part may easily be screwed into a bone or bone part.

A particularly simple construction of the fastening element arises when the fixing-element tool receiver and/or the tool receiver comprises a recess in the form of a polygonal socket or star socket and when the recess is open in proximal direction. For example, by means of a square socket, hexagonal socket or octagonal socket it is then possible to vary the position of either the fixing element or the fastening part or both parts. In particular, conventional tools provided in an operating theatre may be used to insert the fastening element. If the fixing-element tool receiver and/or the tool receiver is designed in the form of a star socket, then Torx® tools for example may be used to insert the fastening element.

It is further advantageous when the connecting element in the fixing position lies directly against the proximal end of the fastening part. This makes it possible to simplify the construction of the fastening element, in particular to minimize the number of parts needed for the fastening element. A fixing of the connecting element to the fastening element may be used simultaneously also to fasten the receiving part to the fastening part. The connecting element is used, so to speak, as a kind of clamping element to achieve a load transfer between the fixing element and the proximal end of the fastening part.

So that simply by adjusting the fixing element a change from the fixing position to the adjustment position and vice versa is possible and at the same time the connecting element may also be fixed by the fixing element on the receiving part, it is advantageous when a clamping element is provided for clamping and fixing the receiving part on the fastening part in the fixing position. For example, by locking a screw serving as a fixing element two fixing operations may be carried out simultaneously, i.e. in one step, namely on the one hand the receiving part may be fastened to the fastening part and on the other hand the connecting element may be fastened to the receiving part.

It is advantageous when the clamping element in the fixing position is held clamped between the fixing element and the connecting element. This development allows the connecting element to press directly against the proximal end of the fastening element, whereas the fixing element presses indirectly via the clamping element upon the connecting element.

According to a preferred form of construction of the invention, it may be provided that the clamping element in the fixing position is held clamped between the connecting element and the proximal end of the fastening part. It is then possible to press the connecting element against the clamping element and to press the clamping element in turn against the proximal end of the fastening part, namely with the fixing element.

So that the fastening part may be supported particularly well relative to the receiving part in the fixing position, the clamping element may have a distal end corresponding to the proximal end of the fastening part.

Particularly in the case of a dome-shaped design of the proximal end of the fastening part, it is advantageous when the distal end of the clamping element defines a hollow-dome-shaped seating surface. This allows the clamping element to lie with the greatest possible surface area in contact with the proximal end of the fastening part.

It is particularly advantageous when the clamping element has a proximal end that has relative to the reference plane a connecting-element seating surface, which is inclined by a clamping-element angle of inclination and forces in proximal direction and against which the connecting element lies in the fixing position. This development makes it possible to press with the fixing element against the connecting element, which lies directly adjacent to the clamping element and presses the clamping element against the proximal end of the fastening part. By virtue of the inclined proximal end of the clamping element, it may be ensured that the connecting element may be fastened to the receiving element at a desired inclination relative thereto.

According to a further preferred form of construction of the invention, it may be provided that the clamping element has a proximal end that has relative to the reference plane an inclined connecting-element seating surface facing in proximal direction, that the connecting-element receiver comprises at least one connecting-element-receiver seating surface facing in proximal direction and that the connecting element in the fixing position lies partially against the connecting-element seating surface and partially against the at least one connecting-element-receiver seating surface and is inclined by the connecting-element angle of inclination relative to the reference plane. In other words, in this development the connecting element lies partially against the connecting-element receiver and partially against the proximal end of the clamping element. In this way, an asymmetrical introduction of force from the fixing element via the connecting element to the connecting-element receiver and the clamping element is realized. As a result of this, forces may be introduced not only parallel to, but also transversely of, the longitudinal axis or axis of symmetry of the fixing element. This additionally allows a marked improvement of a clamping between the clamping element and the proximal end of the fastening part.

To allow the connecting element to lie with the greatest possible surface area in contact with the clamping element, it is advantageous when the angle of inclination corresponds or substantially corresponds to the clamping-element angle of inclination. In combination with an outer surface of the fixing element that is inclined at the same angle, a particularly good support of the connecting element on the receiving part may be achieved.

The clamping element is particularly simple to manufacture when it is of a sleeve-shaped design. It moreover affords free access through a likewise sleeve-like receiving part to the proximal end of the fastening part in order, for example, to fix the fastening element in a bone part.

In principle, the fastening part might be of any shape that allows the fastening part to be fixed in or on a bone or the like. Preferably, the fastening part is designed in the form of a bone screw or a bone hook. The fastening part may then easily be driven or hooked and/or screwed into a bone.

An implant system of the initially described type can be improved according to the invention in that at least one of the at least two fastening elements is one of the previously described fastening elements. By means of the previously described fastening elements a connecting element is fastenable in a desired manner in the region of the cervical vertebrae. In particular, it is thereby possible to achieve a, compared to conventional implant systems, particularly acute angle between a longitudinal direction defined by the fastening part and a longitudinal direction defined by the connecting element.

A particularly simple construction of the implant system arises when the connecting element is a rod or a plate having a rod-shaped portion.

The following description of a preferred form of construction of the invention is used in connection with

the drawings to provide a detailed explanation.

### BRIEF DESCRIPTION OF THE DRAWINGS

The drawings show:

FIG. 1: a sectional view through an implant system according to the invention having two fastening elements according to the invention;

FIG. 2: an enlarged view of the area A in FIG. 1; and

FIG. 3: an enlarged view of an area A in FIG. 1 of a second embodiment of a fastening element.

### DETAILED DESCRIPTION OF THE INVENTION

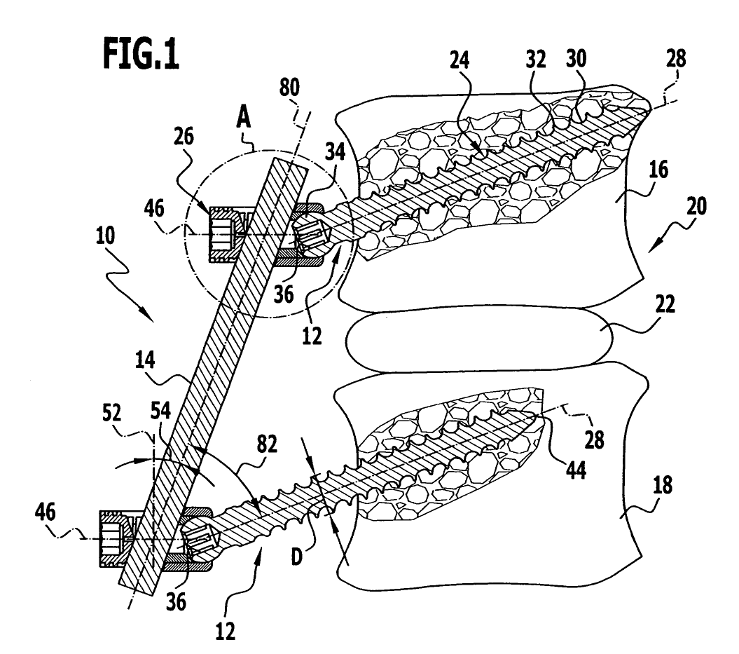

In FIG. 1 an implant system according to the invention provided as a whole with the reference character 10 is illustrated by way of example. In the illustrated embodiment, the implant system comprises two identical fastening elements in the form of polyaxial bone screws 12 as well as a connecting element in the form of a rod 14. By means of the implant system 10 two adjacent vertebrae 16 and 18 of a human or animal vertebral column 20 may be fixed relative to one another. Particularly in the case of damaged cervical vertebrae 16 and/or 18 or a damaged intervertebral disk 22 between them, the implant system 10 may be inserted dorsally for stabilization. The implant system 10 therefore forms a stabilizing system for the vertebral column.

There now follows a detailed description of the construction of the implant system 10.

The rod 14 is elongate and has a circular cross section. It would also be conceivable to use an inwardly curved, non-rectilinearly extending rod 14.

Each of the bone screws 12 comprises a fastening part 24 and a receiving part 26 supported in an articulated manner thereon. The fastening part 24 has an elongate threaded portion 30, which defines a longitudinal axis 28 and extends over approximately ⅘ of the length of the fastening part 24. The threaded portion 30 is provided with an external thread 32 designed as a bone thread. A proximal end 34 of the fastening part 24 is designed in the form of a spherical head, in which a hexagonal socket 36 facing in proximal direction is provided as a tool receiver. Both the head 34 and the hexagonal socket 36 are disposed symmetrically relative to the longitudinal axis 28.

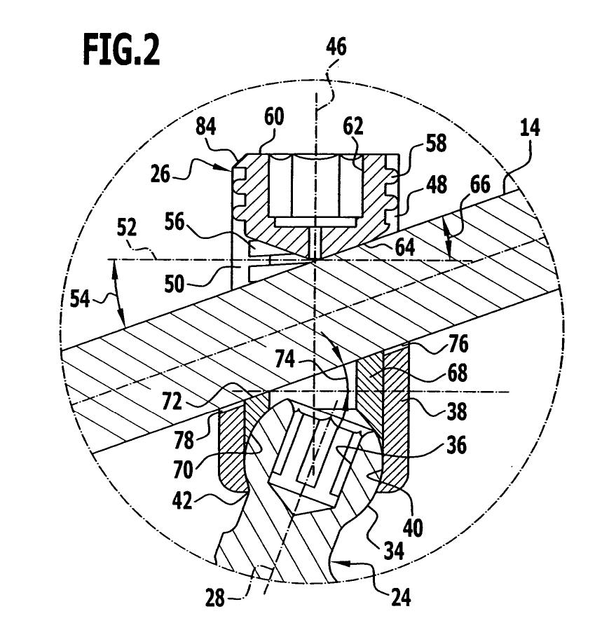

The receiving part 26 is designed in the form of an elongate sleeve 38, which on its distal end has a hollow-dome-shaped seat 40 for the head 34. An inner edge 42 on the distal end of the sleeve 38 has an inside diameter that is smaller than a maximum diameter of the head 34 but larger than a maximum outside diameter D of the threaded portion 30. The fastening part 24 may therefore be inserted, with a tip 44 formed on the distal end of the threaded portion 30 leading the way, from the proximal end through the sleeve 38 until the head 34 lies against the seat 40. Because of the corresponding design of the head 34 and the seat 40, a ball joint connection arises between the fastening part 24 and the receiving part 26. It is therefore possible to pivot the receiving part 26 relative to the fastening part in such a way that a longitudinal axis 46 of the sleeve 38 is inclined relative to the longitudinal axis 28 of the threaded portion 30, as is illustrated both in FIG. 1 and in FIG. 2.

For fastening the rod 14 to the receiving part 26 a connecting-element receiver is provided, which is formed by two mutually diametrically opposite slots 48 and 50 in the sleeve 38. The slots 48 and 50 extend parallel to the longitudinal axis 46, starting from the proximal end of the sleeve 38. The slots 48 and 50 are however of differing depth. The slot 48 extends as far as approximately half of the extent of the sleeve 38 in the direction of the longitudinal axis 46, whilst the slot 50 extends over approximately ¾ of the length of the sleeve 38. Relative to a reference plane 52 extending transversely of the longitudinal axis 46, the connecting-element receiver is therefore inclined by a connecting-element angle of inclination 54, which has a value of approximately 20° to 30°.

The sleeve 38 starting from its proximal end is provided over approximately a third of its length with an internal thread 56, which is of a corresponding design to an external thread 58 of a clamping screw 60 serving as a fixing element. The clamping screw 60 has a hexagonal socket 62 open in proximal direction and is designed as a whole symmetrically relative to the longitudinal axis 46. A distal end of the clamping screw 60 is of a cone-shaped design and has a conical outer surface 64, which is inclined relative to the reference plane 52 by an angle of inclination 66 that corresponds to the connecting-element angle of inclination 54.

Finally, a clamping element in the form of a clamping sleeve 68 is additionally provided, the distal end of which has a hollow-dome-shaped edge 70 of a corresponding design to the head 34 and the proximal end of which is inclined relative to the reference plane 52 by a clamping-element angle of inclination 74 that corresponds both to the connecting-element angle of inclination 54 and to the angle of inclination 66.

In relation to the seat 40 and/or the receiving part 26 and the longitudinal axis 46 thereof, the connecting-element receiver as a whole is designed asymmetrically owing to the two slots 48 and 50 of differing depth. Edges 76 and 78 of the slots 48 and 50 are adapted to the cross section of the rod 14, as is the edge surface 72. The edge surface 72 of the clamping sleeve 68 in fixing position projects in proximal direction slightly beyond the edges 76 and 78. This ensures that the rod 14 may rest on the edge surface 72 but not on the edges 76 and 78.

In order to fasten the rod 14 to the receiving part 26, after introduction of the fastening part 24 into the receiving part 26 the clamping sleeve 68 is introduced, edge 70 first, into the sleeve 38. Then the rod 14 is inserted transversely of the longitudinal axis 46 into the two slots 48 and 50 until it abuts against the edge surface 72. Next, the clamping screw 60 is screwed into the proximal end of the receiving part 26 until the outer surface 64 lies against the rod 14. The clamping screw 60 may then be tightened until the head 34 is held clamped between the clamping sleeve 68 and the seat 40 and at the same time the rod 14 is held clamped between the outer surface 64 and the edge surface 72.

If the position of the receiving part 26 relative to the fastening part 24 is to be varied slightly, the clamping screw 60 merely has to be loosened slightly so that the head 34 is no longer held clamped but is able to be moved relative to the seat 40. Thus, the receiving part 26 as a whole may be pivoted about the centre of the head 34.

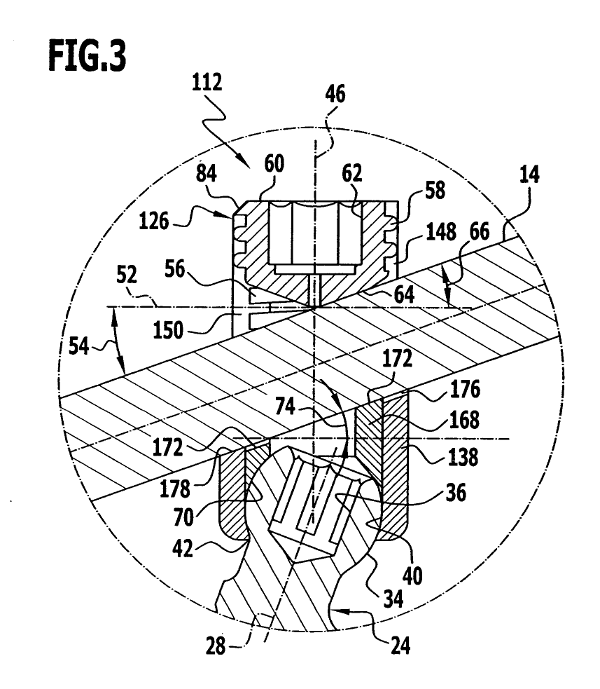

FIG. 3 shows a bone screw, which is provided as a whole with the reference character 112 and is only slightly modified compared to the bone screw 12. Identical parts of the bone screws 12 and 112 are therefore provided with the same reference characters.

The bone screw 112 differs from the bone screw 12 in the design of the connecting-element receiver and the clamping sleeve. The connecting-element receiver in the bone screw 112 is formed likewise by two mutually diametrically opposite slots 148 and 150 in the sleeve 138. The slot 150 is however not quite as deep as the slot 50. On the other hand, the edge surface 72 of the clamping sleeve 168 of the bone screw 112 is inclined to a slightly greater extent relative to the longitudinal axis 46. The rod 14 therefore lies not only on the edge surface 172, but only partially on the edge surface 172, but also simultaneously on the edge 178 of the slot 150. The result is an asymmetrical support of the rod 14 in the connecting-element receiver. As a result of this, the in any case—as in the bone screw 12—asymmetrical introduction of force by the clamping screw 60 at right angles to the outer surface 64 upon the rod 14 and the edge surface 72 and/or a part of the edge surface 172 and the edge 178 is additionally intensified thereby. Thus, the distal end of the clamping sleeve 168, which is designed in the form of a hollow-dome-shaped edge 70, is pressed slightly asymmetrically against the head 34, so that the clamping sleeve 168 may wedge slightly inside the sleeve 138. Thus, both the rod 14 and the sleeve 138 are fastened even more securely to the fastening part.

As the assembly of the bone screw 112 corresponds to that of the bone screw 12, reference is made for this to the above description of the bone screw 12 in connection with FIGS. 1 and 2.

Both the receiving part 26 and the receiving part 126 may be provided at its proximal end with a mark, for example a chamfer 84 as shown in FIGS. 2 and 3, for indicating to a user the direction in which the connecting-element receiver is inclined, i.e. which of the two slots 48 and 50 is deeper than the other. In the two illustrated embodiments, the chamfers 84 are disposed in such a way that they mark the deeper slot 50. As an alternative or in addition to a chamfer, a laser-inscribed mark may be provided, for example at an end face of the sleeve 38 and/or 138 pointing in proximal direction.

The implant system 10 according to the invention allows the adjustment of a minimum pivoting angle 82 between a longitudinal axis 80 of the rod 14 and the longitudinal axis 44 of the fastening part 24, which pivoting angle, compared to conventional implant systems where the rod 14 is held parallel to the reference plane 52, is reduced by the connecting-element angle of inclination 54.

In the implant system 10 illustrated in the drawings, two identical bone screws 12 are provided. It would however also be conceivable to replace one of the two bone screws with a bone screw 112 or with another previously known bone screw that is suitable for fastening the rod 14.

Furthermore, the fastening part 24 may be of any desired design. It would also be conceivable to provide the fastening part 24 in a fixed manner relative to the receiving part 26, in particular it would be possible for both parts also to be of an integral design.

1. Fastening element for an implant system, which comprises at least two fastening elements fixable to bone parts or the like and at least one connecting element fixable to the at least two fastening elements, wherein the fastening element comprises a fastening part with a fastening portion at the distal end as well as a receiving part connected to the fastening part, wherein the receiving part comprises a connecting-element receiver for receiving the connecting element as well as a fixing element for fixing the fastening element in the connecting-element receiver, wherein the connecting-element receiver is designed asymmetrically in relation to an axis of symmetry or longitudinal axis of the fixing element.

2. Fastening element according to claim 1, wherein the connecting-element receiver is inclined by a connecting-element angle of inclination relative to a reference plane extending transversely of the axis of symmetry or longitudinal axis of the fixing element.

3. Fastening element according to claim 1, wherein the receiving part, for forming the connecting-element receiver, has a substantially U-shaped cross section with two free limbs, which are provided with a fixing thread, and the U-shaped cross section is formed by one inclined slot, or by two slots of differing depth, between the free limbs.

4. Fastening element according to claim 1, wherein the fastening part and the receiving part are connected immovably to one another.

5. Fastening element according to claim 1, wherein the fastening part in an adjustment position is adjustable relative to the receiving part and in a fixing position is fixable relative to the receiving part.

6. Fastening element according to claim 5, wherein the fastening part and the receiving part are connected in an articulated manner to one another.

7. Fastening element according to claim 6, wherein an articulated connection is provided between a proximal end of the fastening part and a distal end of the receiving part and the receiving part at the distal end has a receiver for the proximal end of the fastening part.

8. Fastening element according to claim 7, wherein the articulated connection is designed in the form of a ball joint connection and that the proximal end of the fastening part is designed in the form of a spherical-segment-shaped head and the receiver is designed in the form of a dome-shaped seat.

9. Fastening element according to claim 1, wherein the receiving part comprises a fixing thread that co-operates with a fixing-element thread of the fixing element.

10. Fastening element according to claim 6, wherein the fixing thread is an internal thread or an external thread and that the fixing element is a screw or a nut.

11. Fastening element according to claim 1, wherein the receiving part has on its proximal end an open first bore and on the opposite end to the first bore a second bore, the diameter of which is larger than the diameter of a distal end of the fastening part and smaller than the diameter of the proximal end of the fastening part.

12. Fastening element according to claim 11, wherein the edge delimiting the free end of the second bore is designed symmetrically, viewed in relation to a longitudinal axis of the first bore.

13. Fastening element according to claim 1, wherein the receiving part is of a substantially sleeve-shaped design.

14. Fastening element according to claim 2, wherein the fixing element has a tip oriented in distal direction and the tip has an outer surface that is inclined by an angle of inclination relative to the reference plane.

15. Fastening element according to claim 14, wherein a portion or area of the outer surface extends parallel or substantially parallel to the connecting-element receiver.

16. Fastening element according to claim 14, wherein the connecting-element angle of inclination corresponds to the angle of inclination.

17. Fastening element according to claim 14, wherein the angle of inclination has a value in the range of 3° to 50°, in particular a value in the range of 5° to 30°.

18. Fastening element according to claim 1, wherein the fixing element has a fixing-element tool receiver.

19. Fastening element according to claim 1, wherein the fastening part has a tool receiver disposed on the proximal end.

20. Fastening element according to claim 18, wherein the fixing-element tool receiver and/or the tool receiver comprises a recess designed in the form of a polygonal socket or star socket and the recess is open in proximal direction.

21. Fastening element according to claim 5, wherein the connecting element in the fixing position lies directly against the proximal end of the fastening part.

22. Fastening element according to claim 5, wherein a clamping element is provided for clamping and fixing the receiving part on the fastening part in the fixing position.

23. Fastening element according to claim 22, wherein the clamping element in the fixing position is held clamped between the fixing element and the connecting element.

24. Fastening element according to claim 22, wherein the clamping element in the fixing position is held clamped between the connecting element and the proximal end of the fastening part.

25. Fastening element according to claim 22, wherein the clamping element has a distal end corresponding to the proximal end of the fastening part.

26. Fastening element according to claim 25, wherein the distal end of the clamping element defines a hollow-dome-shaped seating surface.

27. Fastening element according to claim 24, wherein the clamping element has a proximal end that has relative to the reference plane a connecting-element seating surface, which is inclined by a clamping-element angle of inclination and faces in proximal direction and against which the connecting element lies in the fixing position.

28. Fastening element according to claim 24, wherein the clamping element has a proximal end that has relative to the reference plane an inclined connecting-element seating surface facing in proximal direction, the connecting-element receiver comprises at least one connecting-element-receiver seating surface facing in proximal direction and the connecting element in the fixing position lies partially against the connecting-element seating surface and partially against the at least one connecting-element-receiver seating surface and is inclined by the connecting-element angle of inclination relative to the reference plane.

29. Fastening element according to claim 27, wherein the angle of inclination corresponds or substantially corresponds to the clamping-element angle of inclination.

30. Fastening element according to claim 22, wherein the clamping element is of a sleeve-shaped design.

31. Fastening element according to claim 1, wherein the fastening part is designed in the form of a bone screw or a bone hook.

32. Implant system having at least two fastening elements fixable to bone parts or the like and having at least one connecting element fixable to the two fastening elements, wherein at least one of the at least two fastening elements is a fastening element comprising a fastening part with a fastening portion at the distal end as well as a receiving part connected to the fastening part, wherein the receiving part comprises a connecting-element receiver for receiving the connecting element as well as a fixing element for fixing the fastening element in the connecting-element receiver, wherein the connecting-element receiver is designed asymmetrically in relation to an axis of symmetry or longitudinal axis of the fixing element.

33. Implant system according to claim 32, wherein the connecting element is a rod or a plate having a rod-shaped portion.
